(19) United States
(12) Patent Application Publication
(10) Pub. No.: US 2012/0277511 A1
(43) Pub. Date: Nov. 1, 2012

(54) High Temperature Platformer

(75) Inventors: Mark D. Moser, Elk Grove Village, IL (US); Clayton C. Sadler, Arlington Heights, IL (US); Mark P. Lapinski, Aurora, IL (US)

(73) Assignee: UOP LLC, Des Plaines, IL (US)

(21) Appl. No.: 13/440,381

(22) Filed: Apr. 5, 2012

Related U.S. Application Data
(60) Provisional application No. 61/480,654, filed on Apr. 29, 2011.

(51) Int. Cl. C07C 5/393; C07C 7/10

(52) U.S. Cl. 585/412; 585/413

(57) ABSTRACT

A process for reforming a hydrocarbon stream is presented. The process involves increasing the processing temperatures in the reformers. The reformers are operated under different conditions to utilize advantages in the equilibriums, but require modifications to prevent increasing thermal cracking and to prevent increases in coking. The process utilizes a common catalyst, and common downstream processes for recovering the desired aromatic compounds generated.

### CROSS-REFERENCE TO RELATED APPLICATIONS

This application claims priority from U.S. Provisional Application No. 61/480,654, filed Apr. 29, 2011, the contents of which are hereby incorporated by reference in its entirety.

### FIELD OF THE INVENTION

The present invention relates to the process of enhancing the production of aromatic compounds. In particular the improvement and enhancement of aromatic compounds such as benzene, toluene and xylenes from a naphtha feedstream through changing process conditions.

### BACKGROUND OF THE INVENTION

The reforming of petroleum raw materials is an important process for producing useful products. One important process is the separation and upgrading of hydrocarbons for a motor fuel, such as producing a naphtha feedstream and upgrading the octane value of the naphtha in the production of gasoline. However, hydrocarbon feedstreams from a raw petroleum source include the production of useful chemical precursors for use in the production of plastics, detergents and other products.

The upgrading of gasoline is an important process, and improvements for the conversion of naphtha feedstreams to increase the octane number have been presented in U.S. Pat. Nos. 3,729,409, 3,753,891, 3,767,568, 4,839,024, 4,882,040 and 5,242,576. These processes involve a variety of means to enhance octane number, and particularly for enhancing the aromatic content of gasoline.

Processes include splitting feeds and operating several reformers using different catalysts, such as a monometallic catalyst or a non-acidic catalyst for lower boiling point hydrocarbons and bi-metallic catalysts for higher boiling point hydrocarbons. Other improvements include new catalysts, as presented in U.S. Pat. Nos. 4,677,094, 6,809,061 and 7,799,729. However, there are limits to the methods and catalysts presented in these patents, and which can entail significant increases in costs.

### SUMMARY OF THE INVENTION

The present invention is a process for improving the yields of aromatics from a hydrocarbon feedstream. In particular, the process converts non-aromatic hydrocarbons in a naphtha feedstream to aromatics in the C6 to C8 range. The non-aromatics include paraffins, olefins and naphthenes. The process improves the yields of aromatics over the currently used methods of processing a naphtha feedstream. The process includes passing the naphtha feedstream to a reformer, wherein the reformer is operated at a temperature greater than 540° C. The operational temperature is equal to the feed inlet temperature, and the reformer comprises a plurality of reactor beds with interbed heaters to maintain the reactor temperature at as uniform a temperature as possible. The reforming process is endothermic, and the temperatures will drop from the inlet temperature due to endothermicity. The reformer generates a process stream comprising aromatics in the C6 to C8 range, and the process stream is passed to a fractionation unit to separate C4 and lighter hydrocarbons from the process stream. The fractionation unit generates a bottoms stream comprising C5 and heavier hydrocarbons. The bottoms stream is passed to an aromatics extraction unit to create an aromatics process stream and a raffinate stream. The process can include the injection of sulfur compounds to limit the amount of coking due to the increased temperature of operation. The process can also utilize a reactor having an internal surface treated to limit coking.

Other objects, advantages and applications of the present invention will become apparent to those skilled in the art from the following detailed description and drawings.

### BRIEF DESCRIPTION OF THE DRAWINGS

FIG. 1 shows the LHSV vs. weight check with added sulfur;

FIG. 2 shows the C8 aromatics increase vs. weight check with sulfur;

FIG. 3 shows the C5+ increase vs. weight check start HOS;

FIG. 4 shows the total aromatics increase;

FIG. 5 shows the hydrogen increase;

FIG. 6 shows the increase in the average reaction block temperature vs. weight check start HOS;

FIG. 7 shows the increase in the average reaction block temperature vs. catalyst life;

FIG. 8 shows the total aromatics increase vs. catalyst life;

FIG. 9 shows the increase in hydrogen vs. catalyst life;

FIG. 10 shows the C5+ increase vs. catalyst life; and

FIG. 11 shows the C8 aromatics increase vs. catalyst life.

### DETAILED DESCRIPTION OF THE INVENTION

Reforming of a hydrocarbon stream for the production of aromatics is an important process. In general, high operating temperatures are preferred for operating a reformer, as the equilibriums at the higher temperatures favors the formation of aromatic compounds. However, the reforming process is operated at a lower temperature due to the thermal cracking and the metal catalyzed coking that occurs as the temperature is increased. It has been found that using reactor vessels with non-metallic coatings allow for higher temperature operations, without the accompanying increase in coking or thermal cracking.

The present invention provides for increased aromatics yields by changing the normal operating parameters for the hydrocarbon reformation process. The reformation process is a process of converting paraffinic hydrocarbons to aromatic hydrocarbons through cyclization and dehydrogenation. The cyclization and dehydrogenation goes through many steps, and can generate olefins as well as naphthenes. In turn the olefins can be cyclized and dehydrogenated, and the naphthenes can be dehydrogenated.

Increasing the temperature would normally be a preferred condition, since the higher temperatures shift the equilibriums of the reforming reactions to favor the production of aromatics. However, increasing the temperatures increases the formation of coke on the catalyst, and more rapidly deactivates the catalyst. Increasing temperatures also increases thermal cracking for the heavier hydrocarbons, and can start or increase metal catalyzed coking on the surfaces of the reactor vessel or piping used to transport the hydrocarbons to the reformer. This in turn requires more energy to regenerate the catalyst on a more frequent basis. Currently, the reformation process has been optimized to run at lower temperatures to balance the production of aromatics against the costs in time and energy of regenerating the catalyst, as well as minimizing thermal cracking and metal catalyzed coking.

The present invention is a process for generating aromatics from a hydrocarbon feedstream. The process includes passing the hydrocarbon feedstream to a reformer, wherein the reformer is operated at a temperature greater than 540C, and the internal surfaces of the reactor are coated with a non-coking material to generate a process stream comprising aromatic compounds. The process stream is passed to a fractionation unit to separate light gas components comprising C4 and lighter hydrocarbons, as well hydrogen and other light gases from the process stream. The fractionation unit generates an overhead stream having the light gas components and a bottoms stream having C5 and heavier hydrocarbons. The bottoms stream is passed to an aromatics extraction unit to create a purified aromatics stream and a raffinate stream having a reduced aromatics content.

The reforming process contacts the hydrocarbon feedstream with a catalyst and performs dehydrogenation and cyclization of hydrocarbons. The process conditions include a temperature greater than 540C, and a space velocity between 0.6 hr-1 and 10 hr-1. Preferably the space velocity is between 0.6 hr-1 and 8 hr-1, and more preferably, the space velocity is between 0.6 hr-1 and 5 hr-1.

The process of the present invention allows for greater heating through altering the reactor surfaces, and the equipment that delivers the heated hydrocarbon feedstream to the reactors. This includes the transfer equipment, such as piping between the fired heaters and the reactor, as well as the internal walls to the surfaces in the fired heaters exposed to the feedstream. The internal surfaces can be sulfide, or coated with non-coking materials, or using a non-coking metallurgy.

In one embodiment, the process for the generation of aromatics from a hydrocarbon feedstream includes heating the hydrocarbon feedstream to a first temperature. The heated hydrocarbon feedstream is passed to a first reformer, which is operated at a first set of reaction conditions, to generate a first reformer effluent stream. The first reformer effluent stream is heated to a second temperature, and the heated first reformer effluent stream is passed to a second reformer. The second reformer is operated at a second set of reaction conditions and generate a second reformer effluent stream. The second reformer effluent stream is passed through a heat exchanger to preheat the feedstream.

The first temperature is a temperature between 500° C. and 540° C., and the second temperature is greater than 540° C. Each reformer can include a plurality of reactors with inter-reactor heaters, wherein each inter-reactor heater heats the stream to a desired temperature, and wherein. For the first reformer, each inter-reactor heater will heat the process streams to the second temperature before passing to the second reformer. With more than two reformers, all reformers except the last one will have the entering process stream heated to the first temperature and the inlet process stream to the last reformer will be heated to the second temperature.

The process can include a tail heater. The tail heater is used to heat the second reformer effluent to a third temperature. The heated second reformer effluent is then passed to a tail reactor. The third temperature is also greater than the first temperature, and preferably is greater than 540C.

The reforming process is a common process in the refining of petroleum, and is usually used for increasing the amount of gasoline. The reforming process comprises mixing a stream of hydrogen and a hydrocarbon mixture and contacting the resulting stream with a reforming catalyst. The usual feedstock is a naphtha feedstock and generally has an initial boiling point of about 80° C. and an end boiling point of about 205° C. The reforming reactors are operated with a feed inlet temperature between 450° C. and 540° C. The reforming reaction converts paraffins and naphthenes through dehydrogenation and cyclization to aromatics. The dehydrogenation of paraffins can yield olefins, and the dehydrocyclization of paraffins and olefins can yield aromatics.

The reforming process is an endothermic process, and to maintain the reaction, the reformer is a catalytic reactor that can comprise a plurality of reactor beds with interbed heaters. The reactor beds are sized with the interbed heaters to maintain the temperature of the reaction in the reactors. A relatively large reactor bed will experience a significant temperature drop, and can have adverse consequences on the reactions. The catalyst can also pass through inter-reformer heaters to bring the catalyst up to the desired reformer inlet temperatures. The interbed heaters reheat the catalyst and the process stream as the catalyst and process stream flow from one reactor bed to a sequential reactor bed within the reformer. The most common type of interbed heater is a fired heater that heats the fluid and catalyst flowing in tubes. Other heat exchangers can be used.

Reforming catalysts generally comprise a metal on a support. The support can include a porous material, such as an inorganic oxide or a molecular sieve, and a binder with a weight ratio from 1:99 to 99:1. The weight ratio is preferably from about 1:9 to about 9:1. Inorganic oxides used for support include, but are not limited to, alumina, magnesia, titania, zirconia, chromia, zinc oxide, thoria, boria, ceramic, porcelain, bauxite, silica, silica-alumina, silicon carbide, clays, crystalline zeolitic aluminasilicates, and mixtures thereof. Porous materials and binders are known in the art and are not presented in detail here. The metals preferably are one or more Group VIII noble metals, and include platinum, iridium, rhodium, and palladium. Typically, the catalyst contains an amount of the metal from about 0.01% to about 2% by weight, based on the total weight of the catalyst. The catalyst can also include a promoter element from Group IIIA or Group WA. These metals include gallium, germanium, indium, tin, thallium and lead.

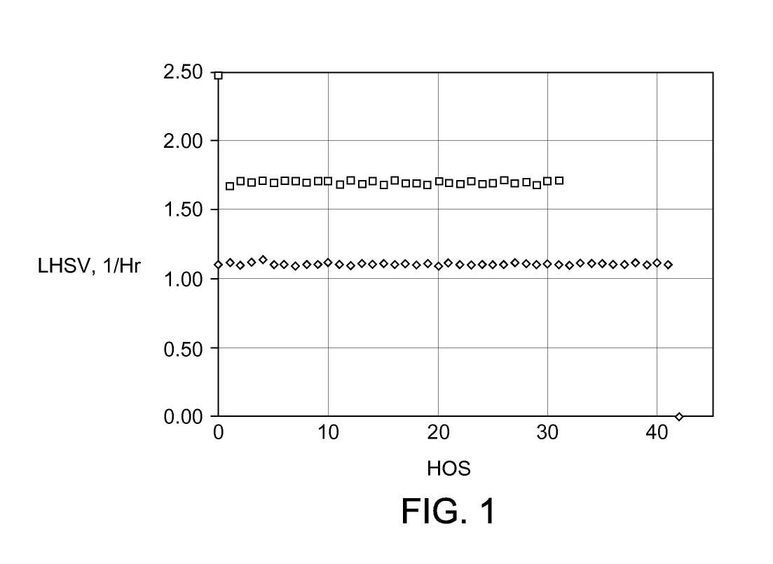

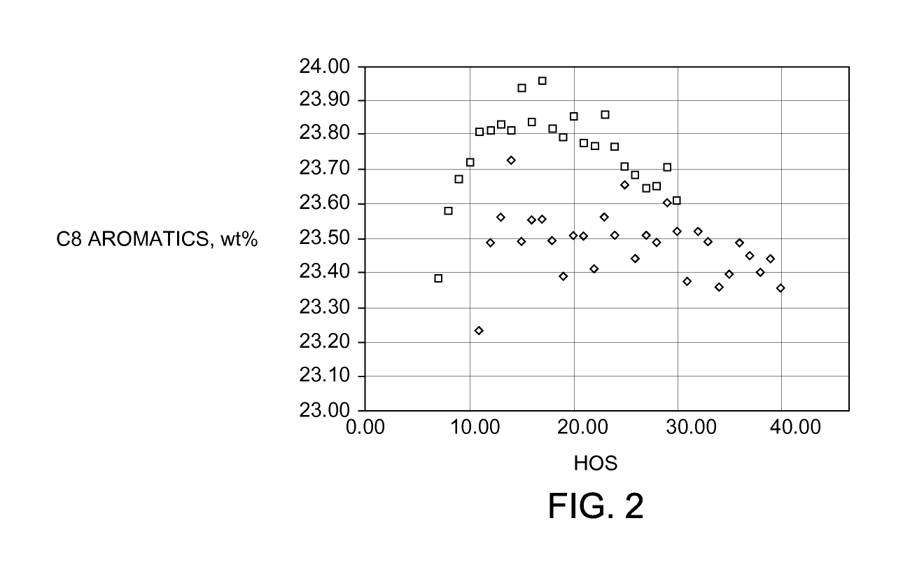

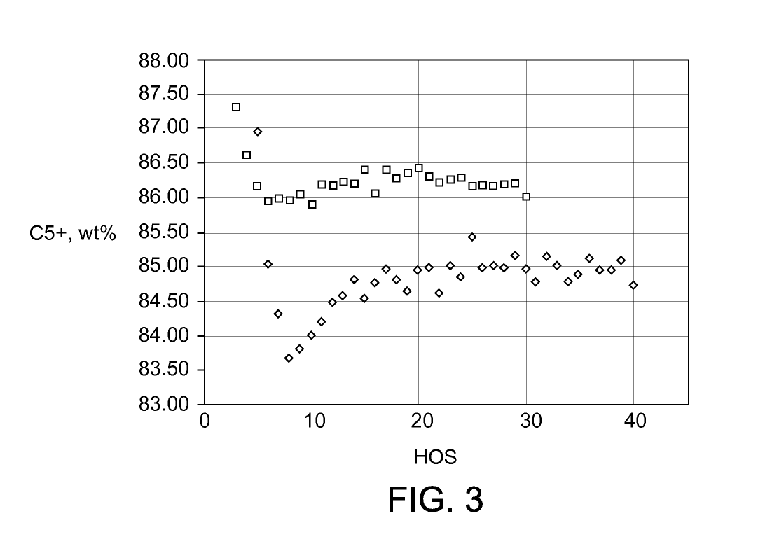

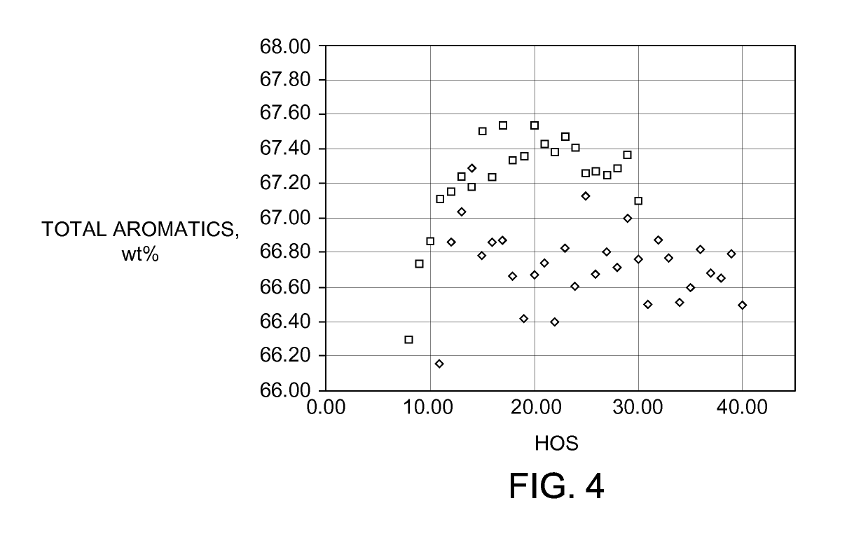

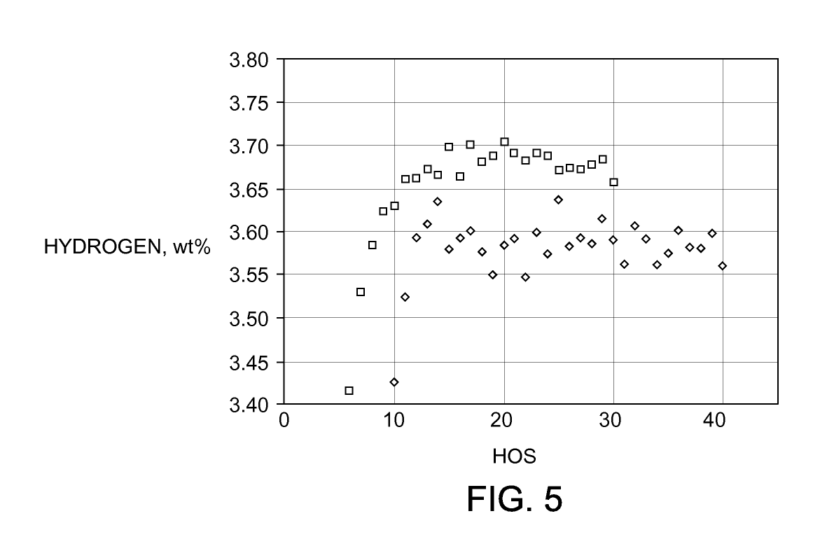

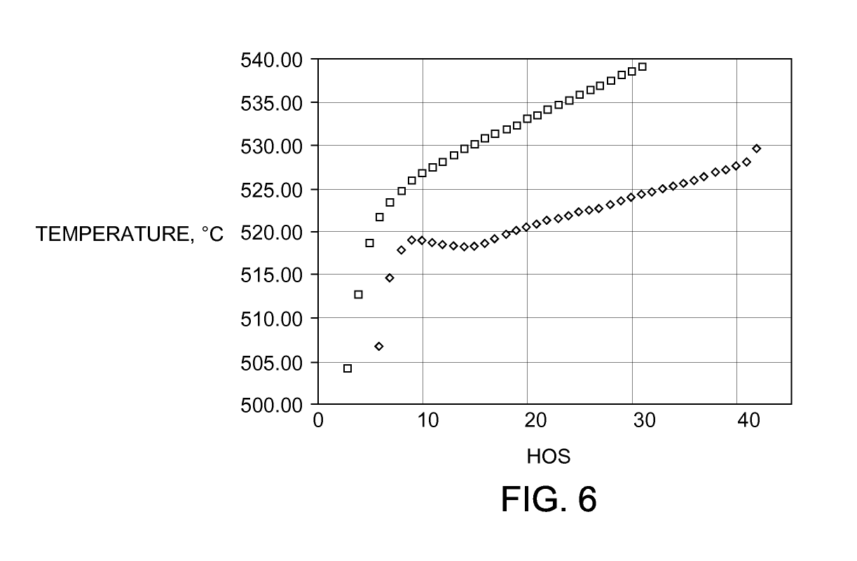

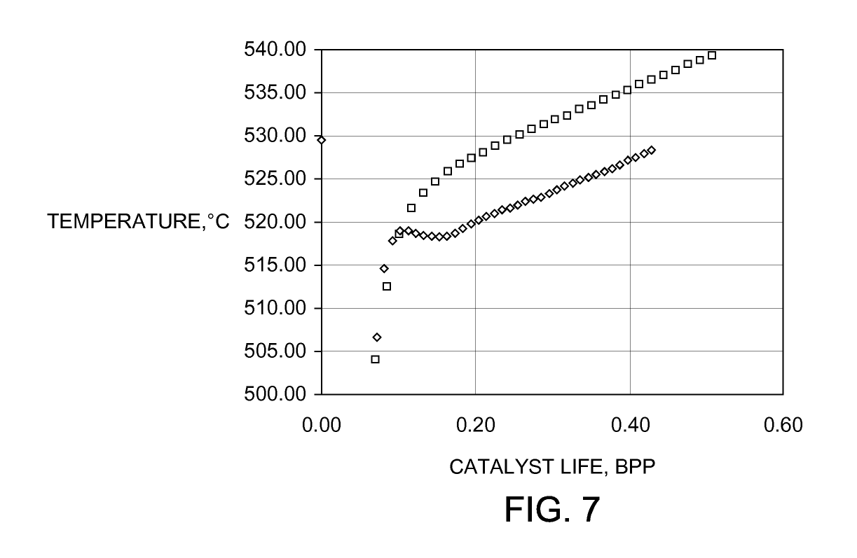

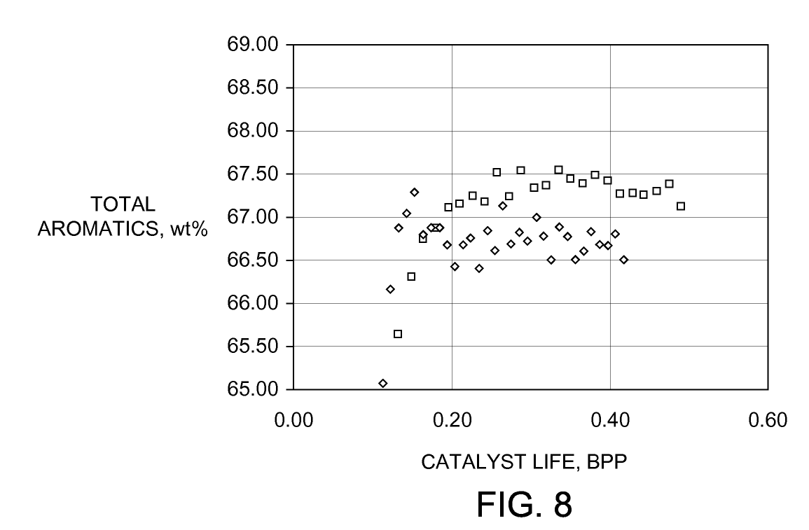

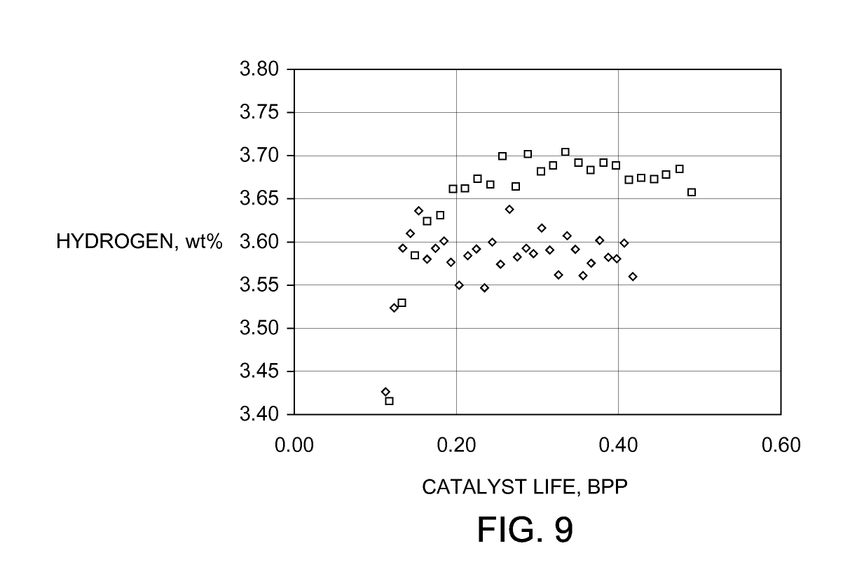

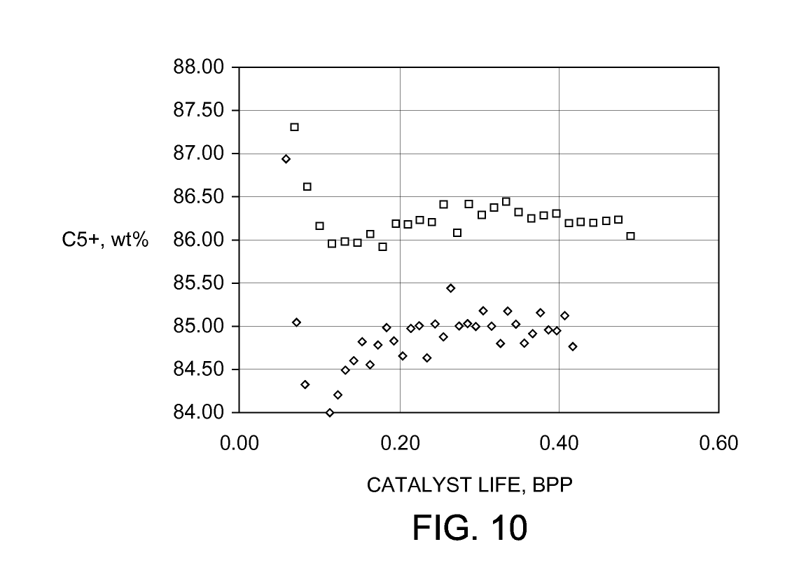

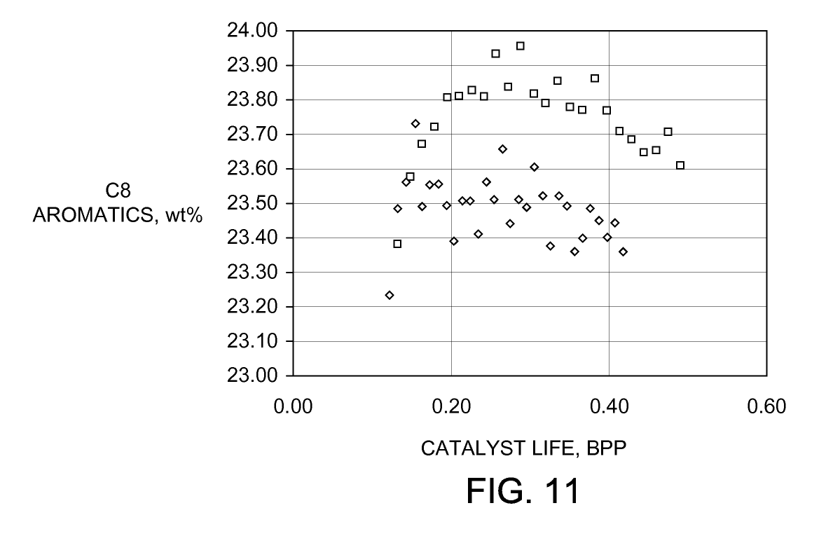

The data, as presented in FIGS. 1-11, shows a significant increase in aromatics, hydrogen and C5+ liquid product when the same catalyst is operated at a higher temperature, but the same catalyst is operated at different space velocities. The experiments with run with a dehydrogenation catalyst, UOP's DEH-5 catalyst, comprising 0.5 wt % Pt, 1.03 wt % Cl on a support. The density of the catalyst was 0.31 g/cc. FIG. 1 shows the weight check with added sulfur during hours on stream (HOS) v. LHSVs of 1.1 (diamonds) and 1.7 (squares). FIG. 2 shows the C8 aromatics increase for the two runs, LHSVs of 1.1 (diamonds) and 1.7 (squares). FIG. 3 shows the C5+ content of the product streams for the two runs, LHSVs of 1.1 (diamonds) and 1.7 (squares). FIG. 4 shows the aromatics increase in the product streams for the two runs, LHSVs of 1.1 (diamonds) and 1.7 (squares). FIG. 5 shows the hydrogen generation during the process for the two runs, LHSVs of 1.1 (diamonds) and 1.7 (squares). FIG. 6 shows the average reaction block temperature for the two runs, LHSVs of 1.1 (diamonds) and 1.7 (squares). FIG. 7 shows the average reaction block temperature vs. catalyst life (BPP), for the two runs, LHSVs of 1.1 (diamonds) and 1.7 (squares). The BPP is a normalized time of operation, or barrels of feed per pound of catalyst. FIG. 8 shows the total aromatics vs. catalyst life for the two runs, LHSVs of 1.1 (diamonds) and 1.7 (squares). FIG. 9 shows the hydrogen produced vs. catalyst life, for the two runs, LHSVs of 1.1 (diamonds) and 1.7 (squares). FIG. 10 shows the C5+ wt. % in the product stream vs. the catalyst life, for the two runs, LHSVs of 1.1 (diamonds) and 1.7 (squares). FIG. 11 shows the C8 aromatics generated in the product stream vs. the catalyst life, for the two runs, LHSVs of 1.1 (diamonds) and 1.7 (squares). This increase is expected at the higher temperature due to a decrease in activity through a reduced chloride content on the catalyst.

The increases due to higher temperatures allow for increased throughputs, or increased federates, and produces more aromatic products at a lower cost.

While the invention has been described with what are presently considered the preferred embodiments, it is to be understood that the invention is not limited to the disclosed embodiments, but it is intended to cover various modifications and equivalent arrangements included within the scope of the appended claims.

1. A process for generating aromatics from a hydrocarbon feedstream, comprising:
passing the hydrocarbon feedstream to a reformer, wherein the reformer is operated at a temperature greater than 540° C., and the internal metal surfaces of the reactor are coated with a non-coking material, thereby generating a process stream comprising aromatics;
passing the process stream to a fractionation unit thereby creating an overhead stream comprising C4 and lighter hydrocarbons, and a bottoms stream comprising C5 and heavier hydrocarbons; and
passing the bottoms stream to an aromatics extraction unit, thereby creating an aromatics process stream, and a raffinate stream.

2. The process of claim 1 wherein the reformer performs dehydrogenation and cyclization reactions.

3. The process of claim 1 wherein the catalyst has a reduced chloride content.

4. The process of claim 1 wherein the space velocity is between 0.6 hr-1 and 10 hr-1.

5. The process of claim 1 wherein the reactor surfaces have been sulfided.

6. The process of claim 1 further comprising using transfer equipment having non-coking coatings or metallurgy, when the transfer equipment is subject to high temperatures.

7. The process of claim 6 wherein the transfer equipment comprises piping.

8. The process of claim 6 wherein the hydrocarbon feedstream is passed through a fired heater prior to the reformer, and the piping has a non-coking coating or metallurgy on the inside pipe wall.

9. A process for the generation of aromatics from a hydrocarbon feedstream comprising
heating the hydrocarbon feedstream to a first temperature;
passing the heated hydrocarbon feedstream to a first reformer, thereby creating a first reformer effluent stream;
heating the first reformer effluent stream to second temperature and passing the heated stream to a second reformer, thereby creating a second reformer effluent stream; and
passing the second reformer effluent stream through a heat exchanger to pre-heat the feedstream.

10. The process of claim 9 wherein the first reformer comprises a plurality of reactors, with inter-reactor heaters, and wherein each reactor has a feedstream, with the feedstream heated to the first temperature by the inter-reactor heaters.

11. The process of claim 9 wherein the first temperature is between 500 and 540° C.

12. The process of claim 9 wherein the second temperature is greater than 540° C.

13. The process of claim 9 further comprising:
passing the second reformer effluent stream through a tail heater to heat the second reformer effluent stream to a third temperature; and
passing the heated second reformer effluent stream to a tail reactor, wherein the third temperature is greater than the first temperature.

14. The process of claim 13 wherein the third temperature is greater than 540° C.
